(19) United States
(12) Patent Application Publication
(10) Pub. No.: US 2023/0165978 A1
(43) Pub. Date: Jun. 1, 2023

(54) Composition and A Method For Diagnosing And Treating Bacterial Infection

(71) Applicant: Hong Kong Centre for Cerebro-Cardiovascular Health Engineering Limited, Hong Kong (HK)

(72) Inventors: Bee Luan Khoo, Hong Kong (HK); Tianfu Zhang, Hong Kong (HK)

(21) Appl. No.: 17/536,193

(22) Filed: Nov. 29, 2021

(51) Int. Cl. A61K 49/00; A61K 41/00; A61K 31/4425; A61K 31/4439; A61P 31/04

(52) U.S. Cl. CPC A61K 49/0052; A61K 41/0057; A61K 31/4425; A61K 31/4439; A61P 31/04

(57) ABSTRACT

The present invention relates to a composition and a method for use in diagnosing and treating bacterial infection. The composition comprises a novel compound with aggregation-induced emission characteristics adapted to target bacterial and biofilm with fluorescence. The compound is further adapted to generate reactive oxygen species for inhibiting and killing bacteria. The compound can be administered as a stand-alone anti-bacterial fluorescent modular probe or in combination with commercial drugs for enhanced therapeutic efficiencies with fewer side effects.

### FIELD OF THE INVENTION

The invention relates to the field of targeting and treating bacterial infection.

### BACKGROUND OF THE INVENTION

Cardiovascular diseases (CVDs) and cancers are the leading cause of death around the world. Bacterial infections outside tissues, with bacteria covering the tissue surface and the formation of biofilms, have gained growing attention among CVD and cancer researchers. They are found to play an important role in interacting with inflammatory and immunological pathways as chemo-drug resistance behaviors. Biofilms are aggregates of bacteria in which bacterial cells are embedded in a self-produced matrix of extracellular polymeric substances adhering to each other and at a surface. Recent clinical studies revealed that multicellular biofilms could cover or infiltrate the interstitial heart or periphery space of blood vessels of patients, causing dysfunction of the heart and inflammation. Studies have shown that high doses of antibiotics and CVD therapeutics are required to treat biofilms and CVDs compared to the patients having CVDs alone during therapy, resulting in severe multi-drug resistance (MDR) in practice.

Fluorescence imaging-guided photodynamic therapy (PDT) is a useful means of theranostics—a combination of diagnostics and therapeutics, due to its superior controllability, selectivity, precision, negligible drug resistance, and low systemic toxicity. In general, a PDT requires that a fluorescent photosensitizer be provided to generate reactive oxygen species (ROS), which destroy the irradiated bacteria such as under irradiation of light. In recent years, several organic photosensitizers have been developed for use in the diagnosis and/or treatment of cancers or bacterial infections. Nevertheless, they are known to suffer from different intrinsic defects, such as the effect of aggregation-caused quenching (ACQ), which significantly weakens the fluorescence intensity in aggregates during long-term tracking due to energy dissipation via non-radiative decay and, therefore, results in low ROS generation. Aggregation-induced emission (AIE), on the other hand, is an opposite phenomenon to ACQ and was firstly reported in 2001 (J. Luo, Z. Xie, J. W. Lam, L. Cheng, H. Chen, C. Qiu, H. S. Kwok, X. Zhan, Y. Liu, D. Zhu, B. Z. Tang, Aggregation-Induced Emission of 1-Methyl-1,2,3,4,5-Pentaphenylsilole. Chem. Commun. 2001, 1740-1741). AIE photosensitizers have become a useful alternative in disease theranostics thanks to their strong fluorescence emission in the aggregated state, excellent photostability, and selective targeting to the specific organism, which assists in simultaneous bioimaging and targeted targeting photodynamic therapy.

Although various AIE photosensitizers have been developed for bacteria and/or cancer treatments in vitro and in vivo, the applications are never straightforward in treating bacterial infections outside tissues. Therefore, traditional AIE photosensitizers are not yet commonly applied in relevant clinical researches. One reason is that bacteria or biofilms covering tissues often lead to inefficient penetration of light into the in-depth tissue, which affects the ROS generation of the AIE photosensitizers. Even though such a situation could be addressed by enhancing the intensity of the light irradiation and/or by increasing the dosage of the photosensitizers, it may cause undesirable side effects to the normal tissue due to overdosing, such as causing unwanted inflammatory responses.

Furthermore, most of the in vitro models currently available in the field of PDT theranostics are based on conventional, two-dimensional (2D) monolayer cell culture, which fails to properly represent the complex three-dimensional (3D) microenvironment of tissues, thus creating unexpected deviations in drug screening and dose-related studies. While the in vivo research provides decent 3D tumor models, the physiological status is still far from comparison with those in the clinical studies.

Therefore, it is desirable to develop a new technique for identifying bacteria and/or biofilm formation and for reducing, inhibiting, or killing bacteria outside tissues at the same time.

### Objects of the Invention

An object of the present invention is to provide a novel composition and/or method for detecting and treating bacterial infection.

Another object of the present invention is to mitigate or obviate to some degree one or more problems associated with known diagnostic or therapeutic techniques for bacterial infection, or at least to provide a useful alternative.

The above objects are met by the combination of features of the main claims; the sub-claims disclose further advantageous embodiments of the invention.

One skilled in the art will derive from the following description of other objects of the invention. Therefore, the foregoing statements of the object are not exhaustive and serve merely to illustrate some of the many objects of the present invention.

### SUMMARY OF THE INVENTION

In general, the invention provides a composition comprising an agent for use in fluorescent imaging and, at the same time, adapted to reduce, inhibit or eliminate bacterial infection and particularly bacterial infection outside tissues. The agent can be provided in the form of a fluorescent molecular probe with aggregation-induced emission (AIE) property for targeting bacterial such as Gram-negative and Gram-positive bacteria, which can be in the form of planktonic bacteria or biofilms. The agent offers excellent selectivity and signal-to-noise ratio for in situ targeting of bacteria or biofilms on tissue surfaces at high accuracy and precision for qualitative and quantitative analysis. Particularly, the agent is adapted to generate a significantly higher amount of reactive oxygen species (ROS) than traditional photosensitizers for reducing, inhibiting, or killing bacteria under the irradiation of light. Therefore, the present invention demonstrates photodynamic therapy (PDT) application on bacteria targeting and treatment with high efficiency, selectivity, and precision. By combining the agent of the present invention as a PDT photosensitizing agent with other chemical therapeutics such as the anti-cancer drug doxorubicin, it is found that both the doses of the PDT agent and the anti-cancer drug required can be significantly reduced, which is greatly beneficial in reducing or avoiding the undesirable side effects of the treatment. Therefore, the present invention can be used as a stand-alone anti-bacterial fluorescent modular probe or in combination with commercial drugs for enhancing therapeutic efficiencies with fewer side effects. The bi-modal theranotic system of the present invention is unprecedented, offering a synergistic effect in resolving the known shortcomings and dilemma between the low penetration depth of the traditional PDT and the unsatisfactory therapeutic side effect of commercial drugs, serving as a powerful anti-bacterial theranostic system for a bacterial infection when associated with other common diseases, disorders or conditions such as, but are not limited to cancers, cancers related conditions or disorders, cardiovascular diseases, respiratory diseases, and digestive diseases, etc. The combined diagnostic and therapeutic effects are demonstrated by an in vitro 3D model showing the reduction or removal of biofilm covering cardiovascular tissues, which promotes and enhances the therapeutic efficiency of commercial drugs for cardiovascular diseases (CVDs).

In a first main aspect, the invention provides a composition for use in diagnosing and treating bacterial infection, comprising a compound or a pharmaceutically acceptable salt thereof, having the structure of Formula (I):

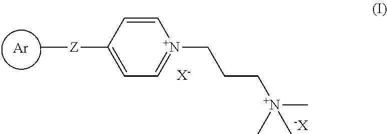

wherein Ar comprises a triphenylamine group or a tetraphenylene group;

Z comprises a direct bond, an electron rich π-conjugated unit, a benzothiadiazole or a benzothiadiazole alkenyl group; and

X comprises a halogen or a derivative thereof.

In a second main aspect, the invention provides a method for diagnosing and treating a bacterial infection in a subject, comprising administering to the subject in need thereof an effective amount of a composition according to the first main aspect.

In a third main aspect, the invention provides a combined diagnostic and therapeutic agent for treating a bacterial infection-associated condition or disorder, comprising the composition according to the first main aspect and one or more therapeutics.

The summary of the invention does not necessarily disclose all the features essential for defining the invention; the invention may reside in a sub-combination of the disclosed features.

### BRIEF DESCRIPTION OF THE DRAWINGS

The foregoing and further features of the present invention will be apparent from the following description of preferred embodiments which are provided by way of example only in connection with the accompanying figure, of which:

FIG. 1A shows the molecular structure of 4-(7-(4-(diphenylamino)phenyl)benzo[c][1,2,5]thiadiazol-4-yl)-1-(3-(trimethylammonio)propyl)pyridin-1-ium bromide (TBPP), an embodied compound of the present invention;

FIG. 1B shows the absorbance of TBPP in DMSO solution (TBPP concentration: 10 μM);

FIG. 1C shows the photoluminescence (PL) spectra of TBPP in DMSO/toluene mixtures with different toluene fractions (TBPP concentration: 10 μM);

FIG. 1D shows the plot of αAIE (I/I₀) versus the different DMSO/toluene mixture of TBPP (TBPP concentration: 10 μM);

FIG. 2 shows the plot of I/I₀ versus light irradiation times in the absence and presence of different photosensitizers (I: fluorescent intensity of dichlorodihydro-fluorescein diacetate (DCFH-DA) in different photosensitizers with different time points after light irradiation. I₀: fluorescent intensity of DCFH-DA in different photosensitizers before light irradiation; concentration of DCFH-DA: 40 μM; concentration of Ce6 and TBPP: 10 μM);

FIG. 3 shows the staining of Gram-negative bacteria by TBPP under different time points (TBPP concentration: 10 μM);

FIG. 4 shows the quantitative study of TBPP by recording the fluorescent intensity according to FIG. 1C;

FIG. 5 shows the staining of uropathogenic Escherichia coli (UPEC) biofilm by TBPP (TBPP concentration: 1 μM);

FIG. 6 shows the staining of human bladder carcinoma cell line (UMUC-3) by TBPP (TBPP concentration: 1 μM);

FIG. 7 shows the staining by TBPP under co-incubation of cancer cells (UMUC-3) and Gram-negative bacteria (UPEC) (TBPP concentration: 1 μM);

FIG. 8 shows the 3D reconstructions of cancer cell clusters comprising bacteria formed in 3D models upon 1 h, 9 h, 24 h after infection (scale bar: 100 μm; concentration of TBPP: 1 μM; concentration of Calcein-AM: 500 nM);

FIG. 9 shows the enlarged 3D imaging of cancer cell clusters comprising bacteria formed in the 3D models 9 h after infection (scale bar; 50 μm; concentration of TBPP: 1 μM; concentration of Calcein-AM: 500 nM);

FIG. 10 shows the enlarged 3D imaging of cancer cell clusters comprising bacteria formed in the 3D models upon 24 h after infection (scale bar: 50 μm; concentration of TBPP: 1 μM; concentration of Calcein-AM: 500 nM);

FIG. 11 shows the 3D imaging of cancer cell clusters and bacteria (scale bar: 100 μm; concentration of TBPP: 1 μM; concentration of Calcein-AM: 500 nM);

FIG. 12 shows the viability of cancer cells in the bacteria outside tissue infected group after doxorubicin (DOX) and TBPP/DOX combinatorial treatment for 24 h. NS=not significant, *** states for p values of <0.001; ** states for p values of <0.01, * states for p values of <0.05;

FIG. 13 shows the changes of morphology and viability of cancer cell clusters in the presence of bacterial inflammation; representative images of clusters stained with Calcein-AM (live cells) and Hoechst (nuclei) after bacteria outside tissue infection for DOX treatment (scale bar: 100 μm);

FIG. 14 shows the changes of morphology and viability of cancer cell clusters in the presence of bacterial inflammation; representative images of clusters stained with Calcein-AM (live cells) and Hoechst (nuclei) after bacteria outside tissue infection for TBPP/DOX combinational treatment (scale bar: 100 μm);

FIG. 15 shows a ¹H NMR spectrum of TBPP; and

FIG. 16 shows a high-resolution mass spectrum of TBPP.

### DESCRIPTION OF PREFERRED EMBODIMENTS

The following description is of preferred embodiments by example only and without limitation to the combination of features necessary for carrying the invention into effect.

Reference in this specification to “one embodiment” or “an embodiment” means that a particular feature, structure, or characteristic described in connection with the embodiment is included in at least one embodiment of the invention. The appearance of the phrase “in one embodiment” in various specifications does not necessarily refer to the same embodiment, nor are separate or alternative embodiments mutually exclusive of other embodiments. Moreover, various features are described, which may be exhibited by some embodiments and not by others. Similarly, various requirements are described, which may be requirements for some embodiments but not other embodiments.

The invention generally relates a composition comprising an agent for use in fluorescent imaging and, at the same time, adapted to reduce, inhibit, or eliminate the bacterial infection, particularly bacterial infection outside tissues. The agent can be provided in the form of a fluorescent molecular probe with aggregation-induced emission (AIE) property for targeting bacterial such as Gram-negative and Gram-positive bacteria, which can be in the form of planktonic bacteria or biofilms. The agent offers excellent selectivity and signal-to-noise ratio for in situ targeting bacteria or biofilms on tissue surfaces at high accuracy and precision for qualitative and quantitative analysis. Particularly, the agent is adapted to generate a significantly higher amount of reactive oxygen species (ROS) than traditional photosensitizers for reducing, inhibiting, or killing bacteria under the irradiation of light. Therefore, the present invention demonstrates photodynamic therapy (PDT) application on bacteria targeting and treatment with high efficiency, selectivity, and precision. By combining the agent of the present invention as a PDT photosensitizing agent with other chemical therapeutics such as the anti-cancer drug doxorubicin, it is found that both the doses of the PDT agent and the anti-cancer drug required can be significantly reduced, which is greatly beneficial in reducing or avoiding undesirable side effects of the treatment. The present invention can thus be used as a stand-alone anti-bacterial fluorescent modular probe or in combination with commercial drugs for enhanced therapeutic efficiencies with fewer side effects. The synergistic effect offered by the bi-modal theranotic system of the present invention is unprecedented, which assists in resolving the known shortcomings and dilemma between the low penetration depth of the traditional PDT and the unsatisfactory therapeutic side effect of commercial drugs, serving as a powerful anti-bacterial theranostic system for a bacterial infection when associated with other common diseases, disorders or conditions such as, but are not limited to cancers, cancers related conditions or disorders, cardiovascular diseases, respiratory diseases, and digestive diseases, etc. The combined diagnostic and therapeutic effects are demonstrated by an in vitro 3D model showing the reduction or removal of biofilm covering cardiovascular tissues, which promotes and enhances the therapeutic efficiency of commercial drugs for cardiovascular diseases (CVDs).

In one embodiment, the present invention provides a composition for diagnosing and treating bacterial infection, including bacterial infection outside tissues. The bacteria may comprise planktonic bacteria or biofilms. The bacterial may comprise Gram-negative and Gram-positive bacteria. Particularly, the composition may comprise a compound or a pharmaceutically acceptable salt thereof, having the structure of Formula (I):

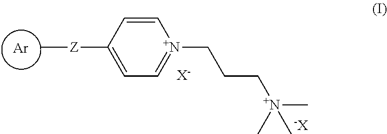

wherein

Ar comprises a triphenylamine group or a tetraphenylene group;

Z comprises a direct bond, an electron-rich π-conjugated unit, a benzothiadiazole, or a benzothiadiazole alkenyl group; and

X comprises a halogen or a derivative thereof.

Preferably, Ar comprises a triphenylamine group selected from a group consisting of:

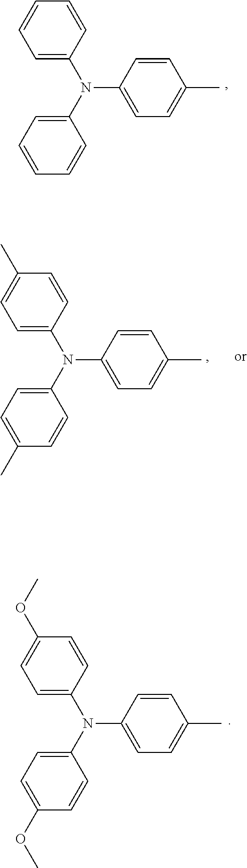 [SMILES: Cc1ccc(N(c2ccccc2)c2ccccc2)cc1 ; Cc1ccc(N(c2ccc(C)cc2)c2ccc(C)cc2)cc1 ; COc1ccc(N(c2ccc(C)cc2)c2ccc(OC)cc2)cc1]

Preferably, Ar comprises a tetraphenylene group selected from a group consisting of:

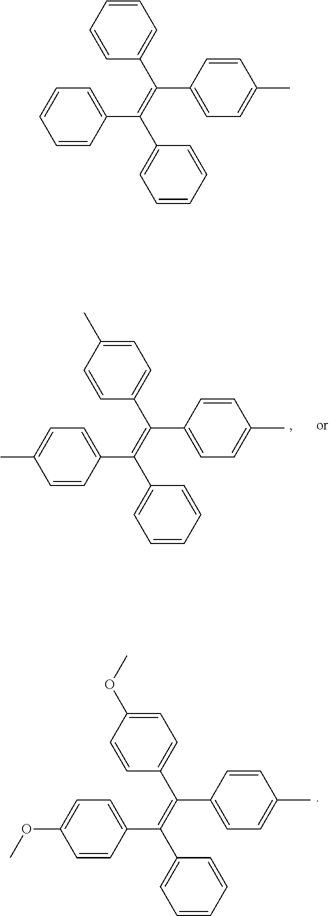

Preferably, Z comprises a benzothiadiazole vinyl group.

Preferably, X comprises bromine or a derivative thereof.

Preferably, the compound of Formula (I) is selected from the group consisting of:

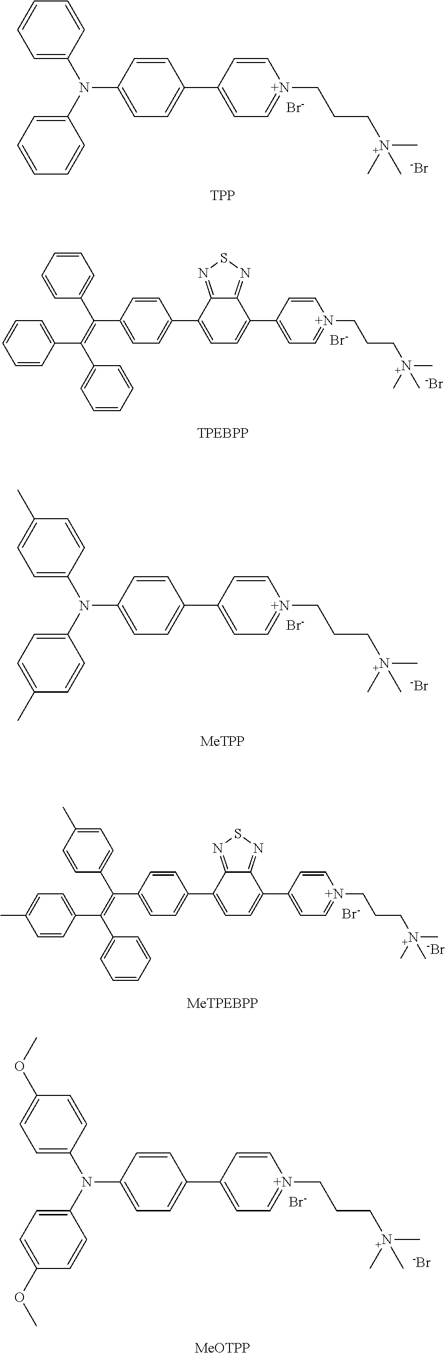

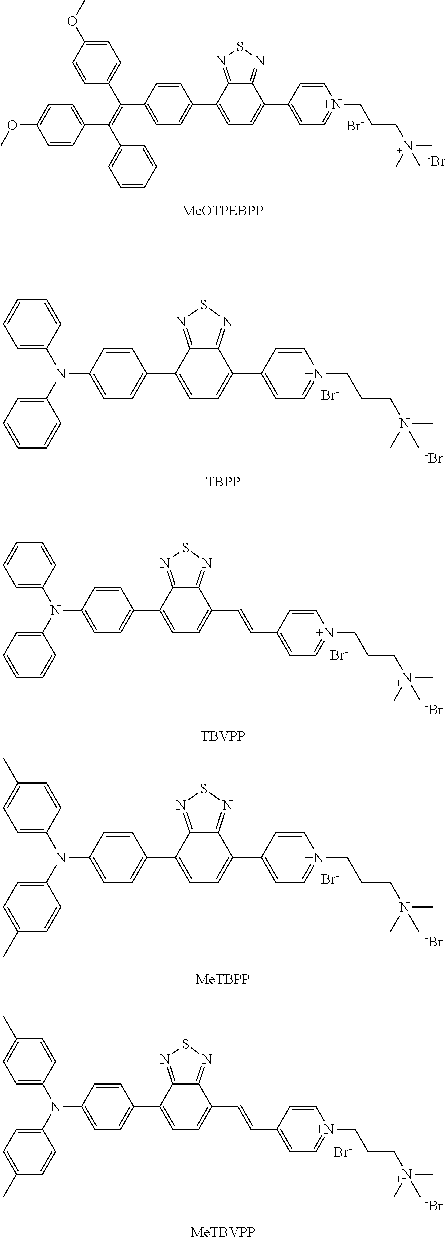

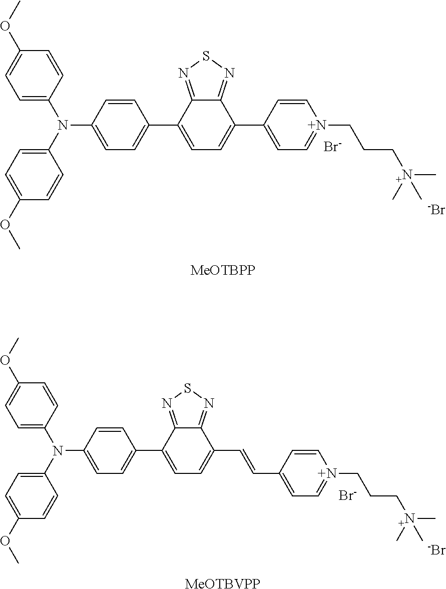

Preferably, the compound of Formula (I) is selected from the group consisting of:
4-(4-(diphenylamino)phenyl)-1-(3-(trimethylammonio)propyl)pyridin-1-ium bromide (TPP);
4-(4-(di-p-tolylamino)phenyl)-1-(3-(trimethylammonio)propyl)pyridin-1-ium bromide (MeTPP);
4-(4-(bis(4-methoxyphenyl)amino)phenyl)-1-(3-(trimethylammonio)propyl)pyridin-1-ium bromide (MeOTPP);
1-(3-(trimethylammonio)propyl)-4-(7-(4-(1,2,2-triphenylvinyl)phenyl)benzo[c][1,2,5]thiadiazol-4-yl)pyridin-1-ium bromide (TPEBPP);
(E)-4-(7-(4-(2-phenyl-1,2-di-p-tolylvinyl)phenyl)benzo[c][1,2,5]thiadiazol-4-yl)-1-(3-(trimethylammonio)propyl)pyridin-1-ium bromide (MeTPEBPP);
(Z)-4-(7-(4-(1,2-bis(4-methoxyphenyl)-2-phenylvinyl)phenyl)benzo[c][1,2,5]thiadiazol-4-yl)-1-(3-(trimethylammonio)propyl)pyridin-1-ium bromide (MeOTPEBPP);
4-(7-(4-(diphenylamino)phenyl)benzo[c][1,2,5]thiadiazol-4-yl)-1-(3-(trimethyl ammonio)propyl)pyridin-1-ium bromide (TBPP);
(E)-4-(2-(7-(4-(diphenylamino)phenyl)benzo[c][1,2,5]thiadiazol-4-yl)vinyl)-1-(3-(trimethylammonio)propyl)pyridin-1-ium bromide (TBVPP);
4-(7-(4-(di-p-tolylamino)phenyl)benzo[c][1,2,5]thiadiazol-4-yl)-1-(3-(trimethyl ammonio)propyl)pyridin-1-ium bromide (MeTBPP);
(E)-4-(2-(7-(4-(di-p-tolylamino)phenyl)benzo[c][1,2,5]thiadiazol-4-yl)vinyl)-1-(3-(trimethylammonio)propyl)pyridin-1-ium bromide (MeTBVPP);
4-(7-(4-(bis(4-methoxyphenyl)amino)phenyl)benzo[c][1,2,5]thiadiazol-4-yl)-1-(3-(trimethylammonio)propyl)pyridin-1-ium bromide (MeOTBPP);
(E)-4-(2-(7-(4-(bis(4-methoxyphenyl)amino)phenyl)benzo[c][1,2,5]thiadiazol-4-yl)vinyl)-1-(3-(trimethylammonio)propyl)pyridin-1-ium bromide (MeOTBVPP).

In one embodiment, the compound of Formula (I) comprises a double charged, pyridinium conjugate having at least one triphenylamine group or at least one tetraphenylethylene group. In one embodiment, Formula (I) compound further comprises a benzothiadiazole or a benzothiadiazole alkenyl group such as a benzothiadiazole vinyl group in the structure.

In one embodiment, the compound of Formula (I) is adapted to emit fluorescence in the wavelengths range from around 550 nm to around 700 nm and preferably exhibits an aggregation-induced emission (AIE) characteristic of fluorescence. This is attributed to the presence of the twisted molecular rotors, i.e., the rotatable phenyl rings in the structure, which exhibit weak or negligible fluorescence in the solution state but can emit an intense fluorescence in an aggregate state or solid state. This specific characteristic allows the compound of Formula (I) to serve as an excellent molecular fluorescent probe with aggregation-induced emission (AIE) characteristic, having high photostability and high signal-to-noise ratio suitable for bioimaging applications.

Formula (I) compound is amphiphilic, having a lipophilic block comprising the triphenylamine or the tetraphenylethylene core and a hydrophilic block comprising the pyridinium moiety with two positive charges. The hydrophilic block provides the compound with good solubility in aqueous media such as water, buffers such as phosphate buffer saline (PBS), and cell culture media such as Dulbecco's Modified Eagle Medium (DMEM), etc. The lipophilic block allows the compound with specific targeting ability for bacterial such as Gram-negative bacteria and Gram-positive bacteria.

In one embodiment, the compounds of TPP, MeTPP, MeOTPP, TPEBPP, MeTPEBPP, or MeOTPEBPP are adapted to emit fluorescence in a visible spectrum ranging from the color green, yellow, orange to red due to the respective functional groups in the molecule. Therefore, these compounds are potentially suitable in applications such as in-vitro imaging and/or for co-staining with different commercially available fluorescence probes.

In one embodiment, TBPP, TBVPP, MeTBPP, MeTBVPP, MeOTBPP, or MeOTBVPP are adapted to emit fluorescence ranging from a visible color red to a near-infrared (NIR) region due to the electron donor-acceptor in the structure. These compounds can exhibit high penetration depth, such as up to about 100 μm during three-dimensional (3D) bioimaging with fewer signal interferences.

In one embodiment, the compound of Formula (I) is adapted to target bacteria outside tissues, such as but are not limited to planktonic bacteria and/or biofilms. In one embodiment, the compound demonstrates selective affinity towards Gram-negative bacteria, e.g., E. coli, Pseudomonas aeruginosa, S. marcescens, and Gram-positive bacteria, e.g., S. aureus and B. subtilis. The selective targeting of bacteria rather than cells further allows visualization of the bacteria growth process on tumors. This bacterial-selective characteristic of the compound could be attributed to the amphiphilic nature and the low partition coefficient between oil and water (about −0.51) of the compound, which effectively prohibits cell penetration but instead demonstrates a preference to anchor at the outer membrane of bacteria. The negative membrane potential of bacteria, generally of about −100 to −120 mV, is known to be much higher than that of cells, generally of about 50 mV, which further attracts the more positively charged compound of Formula (I) via electrostatic interaction.

The compound of Formula (I) is adapted to generate reactive oxygen species (ROS) upon irradiation, such as, but is not limited to, the irradiation of white light. ROS is known to be effective in killing or at least inhibiting bacterial growth. Therefore, bacteria bound with the compound where the light irradiated can be killed efficiently by the ROS generated by the compound. In addition, due to the short lifetime of the generated ROS, which is around 200 ns in an organism, the generated ROS will remain in the irradiated bacteria to exhibit negligible side effects to healthy cells and/or tissue.

In one embodiment, the composition of the present invention may further comprise a therapeutic such as a chemical therapeutic selected from a group consisting of anti-tumor therapeutic, cardiovascular therapeutic, respiratory therapeutic, digestive therapeutic, oral infection therapeutic, and urinary infection therapeutic, etc. For example, the anti-tumor therapeutic may comprise but are not limited to doxorubicin, aldesleukin, cisplatin, oxaliplatin, 5-fluorouracil, cytarabine, gemcitabine, and methotrexate. The cardiovascular therapeutic may comprise but are not limited to hypolipidemic agents (Rosuvastatin), anti-hypertensives (Propranolol, Metoprolol), anti-coagulants (Heparin, Coumadin), etc.

In one embodiment, commercially available drugs such as doxorubicin, an anti-tumor drug, can be administered subsequently or simultaneously after or with the Formula (I) compound to provide a bi-modal diagnostic therapeutic effect to both bacterial infection and cancer treatment. Formula (I) compound may serve as an AIE photosensitizer to target and eliminate bacteria or biofilm covering the infected tissues or the cancerous tissues while or prior to the anti-tumor drug to exhibit its cancer suppression therapeutic effect. At a reduced dosage for both the compound of Formula (I) and doxorubicin, the composition demonstrates an unprecedented synergistic effect which effectively inhibits the bacteria infection, suppresses tumor growth, and at the same time enhances the anti-tumor efficiency of doxorubicin in vitro in a 3D model.

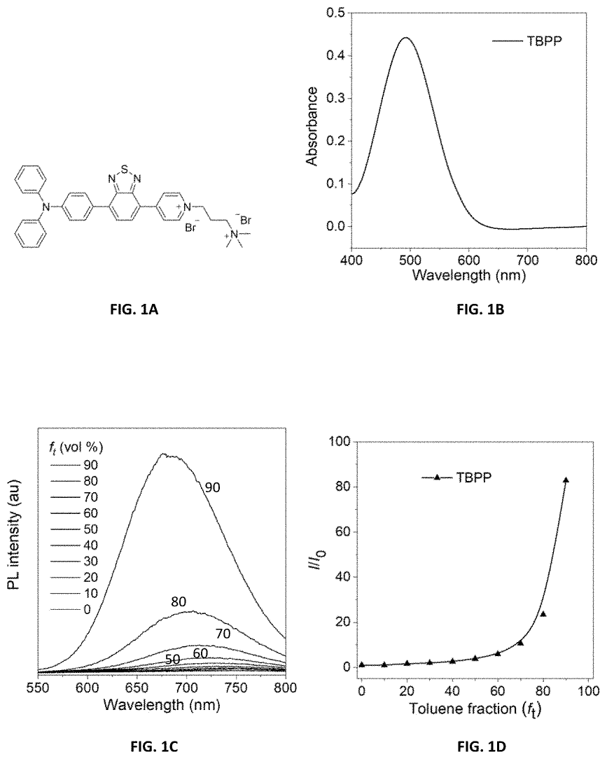

For example, the compound 4-(7-(4-(diphenylamino)phenyl)benzo[c][1,2,5]thiadiazol-4-yl)-1-(3-(trimethyl ammonio)propyl)pyridin-1-ium bromide (TBPP) with the structure shown in FIG. 1A is shown to have significantly reduced the IC50 of doxorubicin for cancer cells (UMUC-3 cell lines) from 8 μM to 5 μM in a 3D model with bacteria outside tissue under 24 hrs of incubation. This combined system has been found to increase the therapeutic effect of doxorubicin at reduced concentration, thus reducing the side effect caused by the therapeutic. TBPP is therefore potentially applicable in removing bacterial infection and biofilms and reducing inflammation at the surface of deep tissues such as tumor tissues, including but not limited to melanoma, lung cancer, and even metastatic cancer and, at the same time, enhancing the efficacy of the administered anti-cancer drugs with fewer side effects.

In addition to the anti-tumor drug of doxorubicin, the bi-modal strategy of the composition comprising the compound of Formula (I) as a PDT photosensitizer, together with chemical therapeutics, can be widely applied in resolving bacterial infection and multi-drug resistance problems during treatment for various diseases such as, but are not limited to, CVDs, respiratory diseases, or digestive diseases, etc. in addition to cancers or cancer-related disease. The composition demonstrated a significant reduction of the IC50 value of common commercial drugs, for example, hypolipidemic agents such as rosuvastatin, anti-hypertensives such as propranolol, and metoprolol; and anti-coagulants such as heparin and coumadin.

The present invention further demonstrates potential applications in treating inflammatory bowel diseases and assisting chemotherapy in deep tissues in colorectal cancer. Furthermore, the present invention can be applied to remove biofilms in cystic fibrosis, to facilitate better internalization of commercial drugs such as elexacaftor, ivacaftor, and tezacaftor for cystic fibrosis, and to reduce viscid mucus and chronic infections to protect lung tissue in the respiratory system and stomach in the digestive system, etc. The present invention further shows the potential to kill the bacteria and inhibit the biofilm formation surrounding the cardiac tissues such as the cardiac valve, coronary heart, myocardial tissue, and peripheral vessel. This assists in recovering the function of the endothelial surface of the valves or inner walls of the arteries and cleans up the infected bloodstreams. Infective endocarditis can also be cured by the enhanced efficiency of CVD drugs with fewer side effects.

The present invention further shows the potential to inhibit biofilm formation surrounding the fatty deposits and calcium accumulated in the arterial wall. The progress of atherosclerosis by lipid secretion from inner cells can be reduced, and specific drugs can remove the fatty deposits and improve the treatment for atherosclerosis. Furthermore, chronic bacterial periodontal diseases are known to be related to hypertension, and particularly stroke. Excess oral bacterial pathogens in both systolic and diastolic blood pressures were shown to be at increased risk for hypertension, reducing the drug efficiency for hypertension therapy. The synergistic therapy system of the present invention thus demonstrates the potential to inhibit chronic bacteria in blood pressures and increase the efficiency of specific drugs such as Diuretics, for example, bumetanide and epitizide, for hypertension.

The compounds of Formula (I) acting as photosensitizers are found to demonstrate excellent bacteria-killing and biofilm removal ability, which can be explored to assist in reducing or curing the bacterial infection in different tissues. For example, the compound can be used in removing the biofilms in the cavity of the middle ear to treat otitis media without the use of broad-spectrum antibiotics and/or tympanostomy tubes which are known to be harmful to patients at a young age. Furthermore, wound infections in integumentary caused by several different microorganisms can be treated by using the anti-bacterial photosensitizers of the present invention. Inflammation can be inhibited through the targeted killing of bacterial biofilm on the wound surface, which helps the wounds to heal faster. Moreover, infection in the respiratory system, such as chronic rhinosinusitis in the paranasal sinuses of the nose and pharyngitis in the throat, can potentially be treated with anti-bacterial photosensitizers of the present invention.

In another aspect of the present invention, it provides a method for diagnosing and treating a bacterial infection in a subject, comprising administering to the subject in need thereof an effective amount of a composition comprising the compound of Formula (I) as described above. Particularly, the method comprises one or more steps of binding the compound of Formula (I) with bacteria comprising planktonic bacteria and/or biofilm; emitting fluorescence by the compound of Formula (I) at the bound bacteria; and/or generating reactive oxygen species (ROS) under irradiation such as the irradiation of light to thereby damage, inhibit the growth of and/or eliminate the bound bacteria by the ROS generated. In one embodiment, the effective amount of the composition comprises around 1 μM to around 10 μM, and preferably, around 1 μM to around 6 μM of the compound of Formula (I). In one embodiment, the irradiation comprises white light at an intensity of around 5 mW/cm² to around 20 mW/cm², preferably around 10 mW/cm².

In one further aspect of the present invention, it provides a method of treating a bacterial infection associated condition or disorder in a subject, comprising the steps of administering to the subject in need thereof an effective amount of the composition comprising the compound of Formula (I) as described above; and subsequently or simultaneously, administering to the subject in need thereof an effective amount of one or more chemical therapeutics. In one embodiment, one or more chemical therapeutics may comprise, but are not limited to, doxorubicin, aldesleukin, cisplatin, oxaliplatin, 5-fluorouracil, cytarabine, gemcitabine, and methotrexate. In one embodiment, the bacterial infection-associated condition or disorder can be one or more of cancer or cancer-related disease, cardiovascular disease, respiratory disease, digestive disease, urinary disease, and oral disease, etc.

### EXAMPLE

### Methodology

### Methods

All chemicals and reagents were commercially available and used as received without further purification. ¹H and ¹³C NMR spectra were measured on a Bruker ARX 400 NMR spectrometer using CDCl₃ and DMSO-d₆ as solvents, and tetramethylsilane (TMS; 6=0 ppm) was chosen as the internal reference. High-resolution mass spectra (HRMS) were obtained on a Finnigan MAT TSQ 7000 mass spectrometer system operated in a matrix-assisted laser desorption and ionization-time-of-flight (MALDI-TOF) mode. Absorption spectra were measured on a Milton Roy Spectronic 3000 array spectrophotometer. Steady-state photoluminescence (PL) spectra were measured on a PerkinElmer spectrofluorometer LS 55.

### Synthesis and Characterization of TBPP

Synthesis of TBPP: TBP was dissolved (100 mg, 0.22 mmol) in 10 mL acetonitrile in a 50 mL two-necked round bottom flask facilitated with a condenser. (3-Bromopropyl)trimethylammonium bromide (Dieckmann, China, 85 mg, 0.33 mmol) was subsequently added and the mixture solution was to refluxed for overnight. After cooling to room temperature, the solution was dried by rotary evaporator and purified by column chromatography (DCM/MeOH=10:1) twice to afford the desired product (100 mg, 63%). ¹H NMR (400 MHz, MeOD-d₄), δ (TMS, ppm): 9.20-9.18 (2H, d), 9.06-9.05 (2H, d), 8.52-8.50 (1H, d), 8.09-8.05 (3H, dd), 7.38-7.34 (4H, t), 7.19-7.13 (8H, m), 4.83-4.81 (2H, t), 3.68-3.65 (2H, m), 3.27 (9H, s), 2.71 (2H, m). HRMS (MALDITOF-MS), m/z: calcd. for C31H25N4S²⁺: 278.6301, found: 278.6289 [M-2Br]²⁺.

### Bacteria Culture

Luria-Bertani (LB) broth (BD Difco, #244620, USA) was chosen for uropathogenic E. coli (UPEC) strain UTI89. A single colony of bacteria on culture medium was transferred to 5 mL of LB and grown at 37° C. for overnight. After reaching the logarithmic phase, a specific amount of bacteria was transferred for further experiments.

### Cell Culture

The human bladder carcinoma cell line UMUC-3 (CRL-1749, ATCC) were cultured in modified Minimum Essential Medium Alpha (MEM a; Gibco, #12561049, USA) supplemented with 10% (v/v) fetal bovine serum (FBS; Gibco, #10270106, USA) and 1% (v/v) penicillin-streptomycin (Gibco, #15140122, USA) at 37° C. with 5% CO2 in a humidified environment. Cells were cultured in sterile T25 or T75 flasks (Jet BIOFIL, China), with growth media replaced every 48 h. Cultures were passaged at 80% confluence.

### ROS Generation Test

Dichlorodihydrofluorescein diacetate (DCFH-DA, Sigma-Aldrich #D6665-5G, 76-54-0, China) was used as the ROS indicator. 40 μL of DCFH-DA stock solution (1 mM) was added into 2 mL of different photosensitizers suspension (10 μM) including TBPPs and chlorin e6 (Sigma-Aldrich #COM448659756-100MG, QC-6466, USA). Then white light (20 mW/cm⁻²) was used to irradiate, and the emission of DCFD-DA at 525 nm was recorded at various irradiation times.

Fabrication of the PIEB Device

The master molds of the tapered microwell layer and the gradient generator for the culture medium were made using diffuser back-side lithography procedures. The tapered microwell was 150×250×150 μm (length×width×depth), and each array contained 300 microwells. The depth of the microchannel was 6 mm. The mold was hard-baked at 150° C. for 5 min. Polydimethylsiloxane (PDMS) molds were made via double-casting. PDMS was prepared using the Sylgard 184 Silicone Elastomer Kit (Dow Corning, USA) via a thorough mixing of the base resin and curing agent in a ratio of 10:1 (w/w). After plasma treatment, the replica PDMS mold was exposed to trichloro (1H,1H,2H,2H-perfluorooctyl) silane (Sigma-Aldrich, #448931, Germany) within a vacuum desiccator for at least 6 h. Polylactic acid (PLA) molds were fabricated for the barrier layer and the gradient generator using 3D printing. The three layers were assembled with plasma treatment for 5 min (high RF level, 700 mmtor), followed by baking for 2 h at 70° C.

### Establishment of Bacteria Outside Tissue Model Via Coating Method

70% (v/v) ethanol (EtOH; UNI-CHEM) was added to channels to sterilize the platform and remove air bubbles. 1× phosphate-buffered saline (PBS; Gibco, #10010049, USA) was used to remove residual EtOH, and the microwell layer was then coated with 50 μL of 2.5% bovine serum albumin (v/v) (BSA) (Sigma-Aldrich, #A9418-5G, Germany) to prevent cell adhesion to the channels. The capacity of each channel was 150 μL. UMUC-3 cell clusters in the microchannels could be stained in situ with Hoechst dye (Invitrogen, #H1399, USA) for 30 min to visualize cell nuclei or biofilm. Imaging and downstream analysis were carried out in situ after 1 h, 9 h, and 24 h of infection. UMUC-3 cell clusters without bacteria were used as negative controls. The bacterial suspension was centrifuged for 10,000 rpm. LB supernatant was removed and replaced with antibiotic-free MEM a (10% supplemented with FBS) at the required multiplicity of infection (MOI) rates (500:1 or 1:1). Control groups corresponding to 0 h time points were obtained before the introduction of bacteria.

### Fluorescent Imaging of Bacteria by TBPP

A single colony of UPEC on solid culture medium was transferred to 5 mL of Luria-Bertani (LB) medium and incubated at 37° C. for overnight. After reaching the logarithmic phase, bacteria were harvested by centrifuging at 10000 g for 3 min. After removing the supernatant, bacteria were diluted in ABTGC (ABT minimal medium supplemented with 2 g 121 glucose and 2 g I21 casamino acids) to 1×105/mL. 300 μL of bacteria solution were added to the culture dish. Then, 300 μL dye solution in saline at appropriate concentration was added into the culture dish for 1 h and directly imaged by Leica TCS SP8 MP confocal microscope (Germany).

### Fluorescent Imaging of IB and Cancer Cells in the Model

1 μm of TBPP and 10 μM of Calcein-AM were added to the channels. Clusters were then imaged after 60 min incubation at 37° C. Z-stack were recorded via confocal microscope.

### Cell Viability Test

Nuclear dye Hoechst (blue) and Calcein-AM (green) Invitrogen, #C3100MP, USA) were added to the channels at a final concentration of 20 μM. Clusters were imaged after 30 min incubation at 37° C. Using ImageJ for analysis. We enumerated only cancer cells within the area range of 25-250 μm² and circularity 0.3-1.0 to ensure that smaller bacteria were excluded from the count.

### Drug Treatments of Biofilms and Cancer Cell Clusters

UMUC-3 cells were seeded into a PIEB device at 35×103 cells/channel concentration and cultured at 37° C. overnight. Then UPEC was suspended in PBS and added into each channel at the MOI of 1:1. Then the bacteria and cells were incubated at 37° C. for 1 h in the device. TBPP was added into the channel and followed by light treatment for 30 mins. The medium was replaced by fresh RPMI with DOX (Sigma-Aldrich, #D1515-10MG, 25316-40-9, China). After 24 h incubation, cells were dyed with Hoechst and Calcein AM for imaging.

### Statistical Analysis

The results were expressed as means±standard deviation. Data groups were compared using the one-way ANOVA and Student's t-test to evaluate associations between independent variables, and the P values were obtained. Three independent trials were conducted in triplicates for each experiment.

### Result

### Photophysical Properties of the Compound of Formula (I)

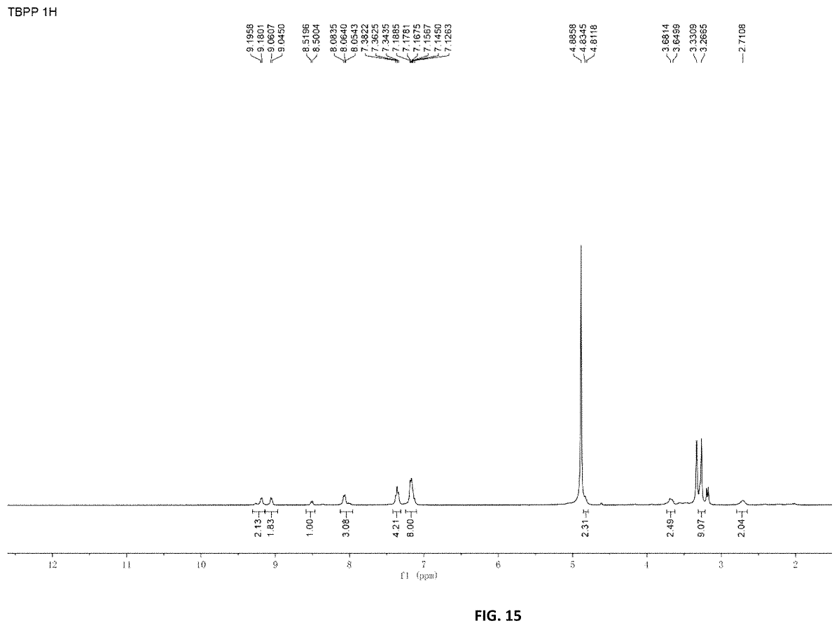

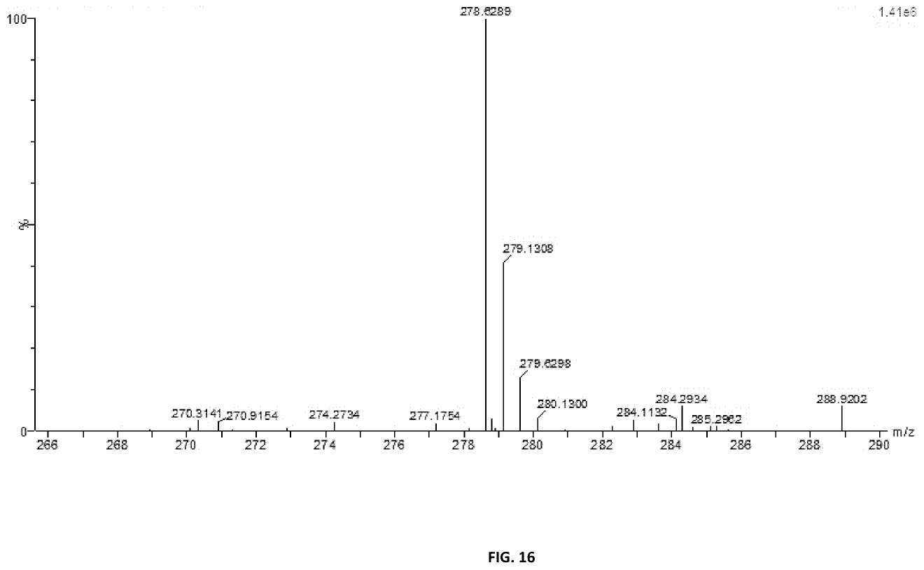

The photophysical properties of an embodied compound, 4-(7-(4-(diphenylamino)phenyl)benzo[c][1,2,5]thiadiazol-4-yl)-1-(3-trimethylammonio)propyl)pyridin-1-ium bromide (TBPP) were evaluated after synthesis and fully characterized with NMR and HRMS (see FIGS. 15 and 16). As shown in the absorption spectra of TBPP in DMSO, it exhibited an absorption maximum at 492 nm (see FIG. 1B), attributed to intramolecular charge transfer transition from the electron-donating triphenylamino group to the pyridinium electron-accepting group. The emission maximum of TBPP in the same condition is 690 nm (see FIG. 1C), which is located at the near-infrared (NIR, 600-1000 nm) region and is ideal for biological application due to the deeper penetration.

The aggregation-induced emission (AIE) property of TBPP was studied in DMSO/Toluene mixtures with different toluene fractions (ft) (see FIG. 1C). In pure or in DMSO solutions, TBPP displayed weak emission. Upon gradually increasing the toluene fraction from ft=0 to 60, the fluorescence intensities of the compounds in the mixture solvent remained weak (see FIGS. 1C and 1D). Further increasing the toluene fraction results in a remarkable fluorescence enhancement, revealing the AIE characteristic of TBPP. In this process, the enhanced emission could be due to the formation of aggregates in the solution, which restricts the intramolecular motions and blocks the non-radiative decay. Furthermore, it is found that TBPP has a large Stokes shift (Δv=198 nm), which is suitable for biological imaging due to the minimized interference between excitation and emission.

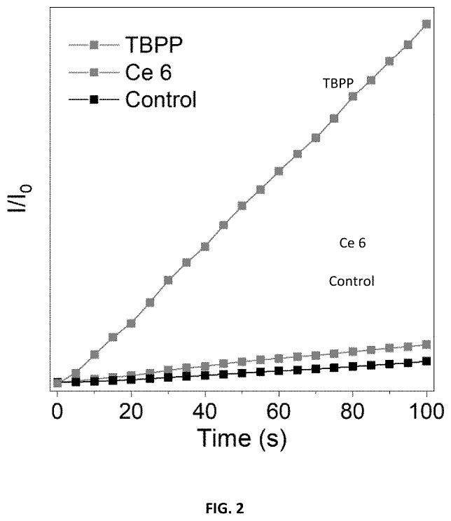

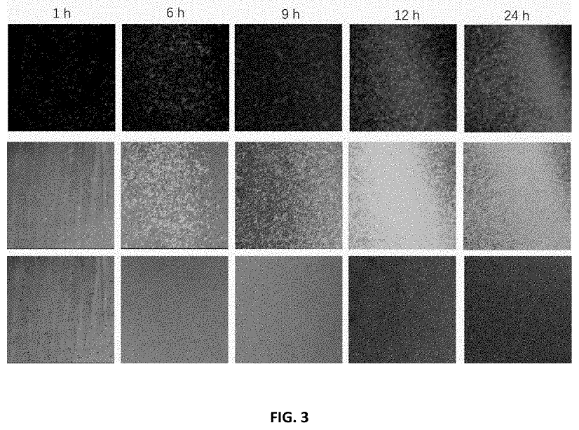

TBPP exhibited efficient ROS generation upon light irradiation. The ROS generation was evaluated under white light irradiation with a commercially available ROS indicator HDCFH-DA because TBPP showed strong absorption in the visible light region. As shown in FIG. 2, the green fluorescence from oxidized DCFH-DA was intensified with increasing irradiation time in the presence of TBPP and showed a much greater change than commercial photosensitizer, Ce6. The good photophysical properties and high ROS generation ability of TBPP demonstrated that TBPP could be used as a potent fluorescent imaging-guided photosensitizer for PDT.

### Monolayer Bacteria Imaging by the Compound of Formula (I)

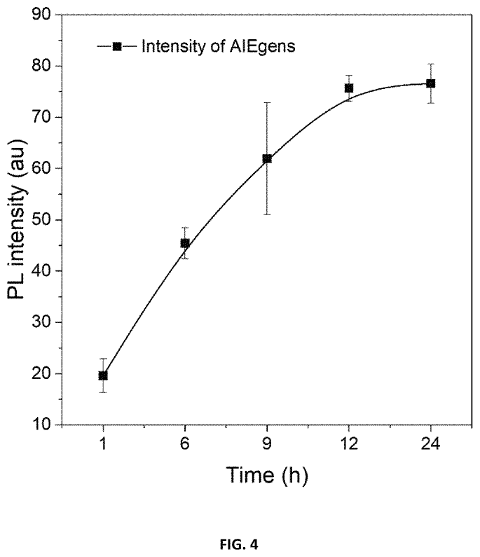

To evaluate the ability of TBPP for monitoring the microbial metabolic status, Gram-negative bacteria UPEC were applied as a representative model in the imaging experiments. UPEC were incubated in a culture dish for a different time, allowing it to grow in the logarithmic growth period upon a monolayer model. As shown in FIG. 3, the near-infra-red (NIR) fluorescent signal of TBPP could be seen from the image, which indicates the targeting ability to UPEC. In addition, The NIR fluorescent intensity of TBPP was increased according to the growth of UPEC during time progress from 1 h to 24 h. As we can see, the fluorescent signal increased linearly from 1 h to 9 h, and the intensity is proportional to the present time, suggesting an exponential phase of UPEC growth. When the bacteria were cultured for more than 12 h, the fluorescent intensity enhancement became slower and gradually reached a plateau in 24 h. This stationary phase is often due to some growth-limiting factors of crowded bacteria, such as the lack of an essential nutrient and the formation of an inhibitory secretion, organic acid. Therefore, the fluorescent bacteria-specific targeting ability of TBPP realizes the quantitative analysis of UPEC (FIG. 4).

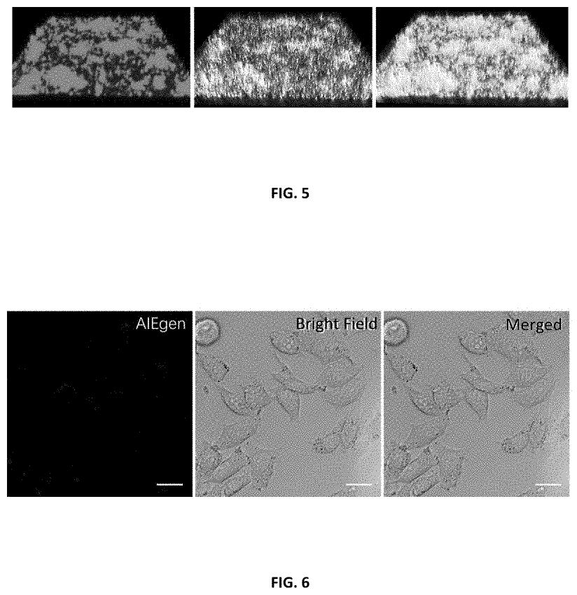

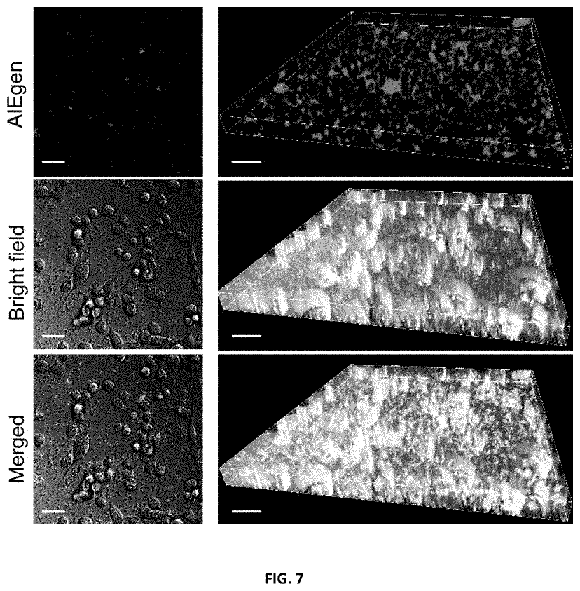

Bacterial biofilm refers to the formation of bacterial aggregate covered with a complicated extracellular matrix. Biofilm endows bacteria with different behaviors and functions, such as antibiotic resistance, strong connections, and tumor progression. Therefore, there is a high demand for the visualization of its structure. As demonstrated, UPEC biofilm was cultivated and selected for the experiment. In FIG. 3, the biofilm morphologies were depicted by the compound TBPP, showing a much higher signal than the planktonic bacteria. Moreover, a loose morphology was observed from the NIR fluorescent intensity of the compound, which attributed to a characteristic of UPEC biofilm. By comparison, the cancer cell staining experiments as shown in FIGS. 5 to 7 exhibited negligible fluorescence, which suggests the molecules have extraordinary selectivity for bacteria over cells.

### 3D Model Imaging of Bacteria Outside Tissue by the Compound of Formula (I) Based on a Microfluidic Platform

A well-established bacteria-tissue model using bladder cancer cells under defined conditions is used as the representative 3D model for the following bioimaging (Y. Deng, S. Y. Liu, S. L. Chua, B. L. Khoo, The effects of biofilms on tumor progression in a 3D cancer-biofilm microfluidic model. Biosens Bioelectron. 2021, 15, 113113). A seeding concentration of 3.5×104 bladder cancer cells (UMUC-3) were uniformly suspended in the growth medium and seeded into each microchannel with multiple microwells, which could produce suitable cell clusters (around 35 cells per microwell; around 8112.46±921.99 μm2) in each microwell with high cell viability. UPEC strain with bacteria: cancer cell ratio (MOIs) of 100:1 was introduced via the coating method to construct the bacterial infection model outside tissue. Bacteria outside tissue by coating method remains on the surface of the tumor clusters, and the biofilms were established at the periphery of the tumor.

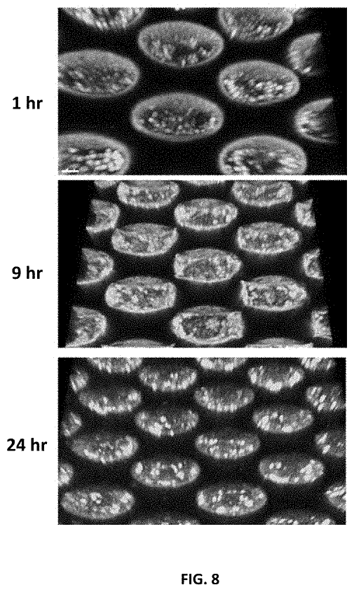

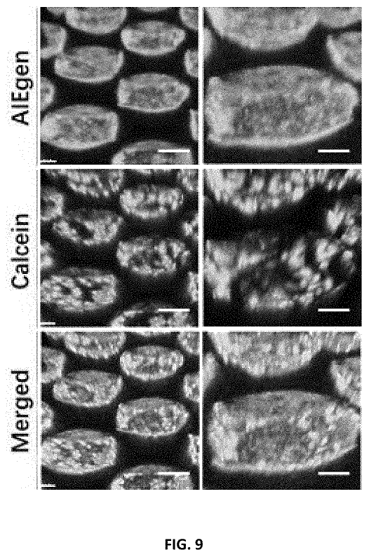

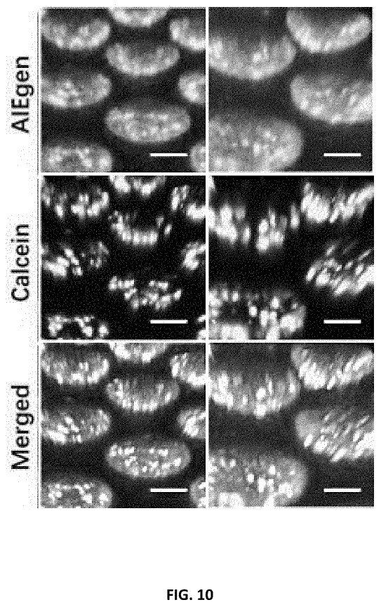

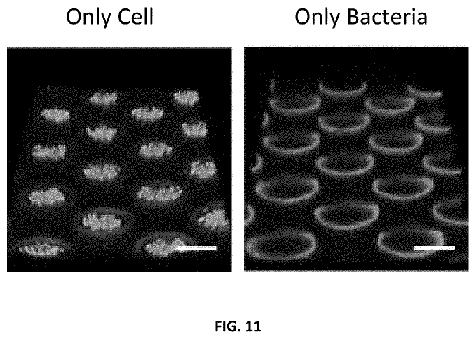

1 μM of TBPP was applied by co-staining with live cells indicator to attempt the visualization of bacteria outside tissue in the 3D microfluidic platform, Calcein-AM, which contained bacterial infection outside the tissue with MOIs of 100:1 and different time points (1 h, 9 h, 24 h) (see FIG. 8). It is found that the NIR fluorescent signal of TBPP spread over the wells under 1 h after infection, indicating the planktonic state of bacteria at the beginning. After 9 h infection, the size of the cancer cell cluster decreased by observing the green fluorescence of Calcein-AM (see FIG. 9). At the same time, the red fluorescent signal covered the periphery of cancer cell clusters, showing the biofilm-forming by bacteria outside tumor tissue. After 24 h co-incubation, bacteria outside tissue and larger biofilm did not result in significant cancer cell killing and exhibited a cancer cell cluster protection ability (see FIGS. 10 and 11). Together with the 3D model, the bacteria targeting TBPP provides an in situ monitoring system of bacteria outside tissue that broadens the horizon in anti-cancer applications.

Combinational Photodynamic/Chemotherapy with the Compound of Formula (I) and Anti-Cancer Drug

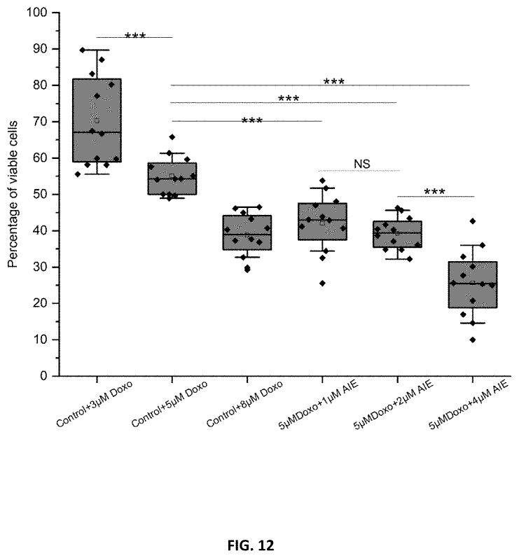

Studies have shown that bacteria outside tissues and biofilm embedded above cancer cell clusters could protect cancer cells as a shield that promotes tumor progression. TBPP is adapted to generate a large amount of ROS under light irradiation and can be used as a photosensitizer to target and kill Gram-negative bacteria efficiently. Therefore, we proposed a combined photodynamic/chemotherapeutic agent comprising the compound of Formula (I) such as TBPP and anti-cancer drug doxorubicin (DOX) to eradicate resident biofilms and cancer clusters simultaneously. As a control, we used doxorubicin solely to evaluate the anti-cancer effect on the model (see FIG. 12). Under 24 h of incubation, the viability of cancer cells treated with 5 μM of TBPP is about 60%.

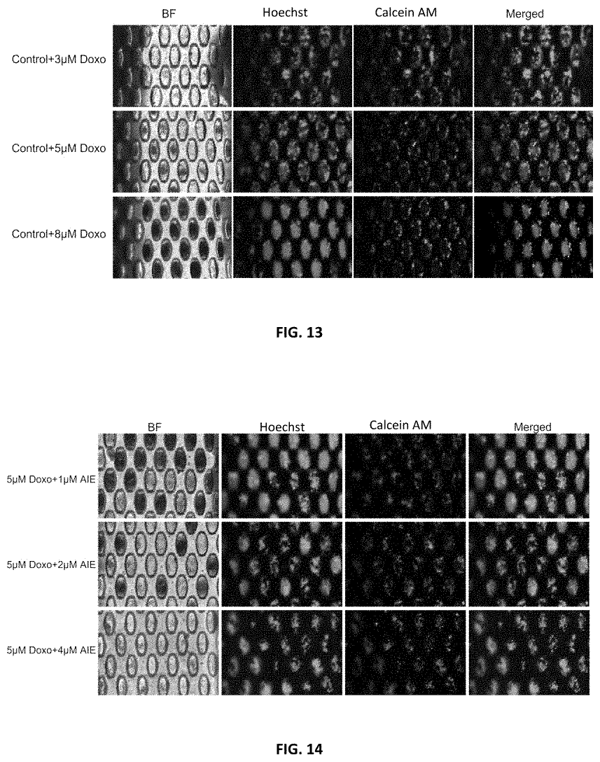

In contrast, it is observed that the viability of the cancer cells treated with TBPP/DOX (5:2, 5:4) was significantly lower than only being treated with doxorubicin, which is significantly lower than 50%. This demonstrates a decrease in IC50 of doxorubicin under the combined therapy. Furthermore, the biofilm was eliminated by the combined therapeutic method with TBPP/DOX (see FIGS. 13 and 14). This further confirmed that the combined photodynamic/chemotherapy strategy of the present invention could reduce drug resistance of cancer cells by destroying the biofilm.

Bacterial infections, including planktonic bacteria and biofilm, are closely related to different diseases in the organs and tissues of a human body, including the auditory, cardiovascular, and urinary systems. Infective endocarditis is the infection in endocarditis by biofilm located on the cardiac valve that can physically interrupt valve function and lead to leakage during the closure of the valve. In addition, biofilm may affect circulation, making the organs such as the brain, kidneys, and heart vulnerable to emboli. Studies have shown that treatments with commercial antibiotics are often unsatisfactory to kill the bacteria unless by prolonged intravenous administration. In this case, surgical excision and replacement of the infected valve are needed, which is costly and particularly harmful to the patient. On the other hand, bacteria and biofilm were also found in the atherosclerotic plaques, which deteriorate the condition by covering the fatty deposits in the arterial walls. Sudden rupture of the plaque can be caused by crowded biofilm, which can be life-threatening. Furthermore, bacteria and biofilm that cover the inner walls of arteries may act as shields to hamper the internalization of drugs and thus hinder the treatment efficiency of the drugs.

The present invention effectively kills bacteria outside tissues and removes biofilms covering valves or artery walls by combining the powerful bactericidal photosensitizers with commercial drugs. The internalization percentage of drugs is therefore promoted. For example, Rosuvastatin is a common drug helping lower bad fats in the blood and decreases the risk of heart diseases and atherosclerotic lesions. However, the drug efficiency would be compromised significantly if bacteria and biofilm are found to cover the lesion or lipid deposits on atherosclerosis because most of the drugs are used to fight against the infections. Secondly, the crowded biofilm covering the surface or inner walls of arteries tends to block the internalization of drugs in the root cause. By combining the bacteria-killing photosensitizer of the present invention with rosuvastatin, bacterial infection on the surface of tissues can be efficiently ablated with minimized side effects and high spatiotemporal precision. The Hypolipidemic agent, rosuvastatin, can lower the lipid in the liver, blood vessels, and heart in a lower dose. In conclusion, the combined therapy is advantageous in offering a bacteria-killing photosensitizer that, when used together with commercial drugs, improves the efficiency of drugs by inhibiting growth of bacteria and biofilm covering the infected tissue surface and thus reducing or minimize the growth of bacteria and biofilm and side effects of the therapy.

While the invention has been illustrated and described in detail in the drawings and foregoing description, the same is to be considered as illustrative and not restrictive in character, it being understood that only exemplary embodiments have been shown and described and do not limit the scope of the invention in any manner. It can be appreciated that any of the features described herein may be used with any embodiment. The illustrative embodiments are not exclusive of each other or other embodiments not recited herein. Accordingly, the invention also provides embodiments that comprise combinations of one or more of the illustrative embodiments described above. Modifications and variations of the invention as herein set forth can be made without departing from the spirit and scope thereof. Therefore, only such limitations should be imposed as indicated by the appended claims.

In the claims hereof, any element expressed as a means for performing a specified function is intended to encompass any way of performing that function. The invention as defined by such claims resides in the fact that the functionalities provided by the various recited means are combined and brought together in the manner the claims call for. It is thus regarded that any means that can provide those functionalities are equivalent to those shown herein.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

It is to be understood that, if any prior art publication is referred to herein, such reference does not constitute an admission that the publication forms a part of the common general knowledge in the art.

1. A composition for use in diagnosing and treating bacterial infection, comprising a compound or a pharmaceutically acceptable salt thereof, having the structure of Formula (I): 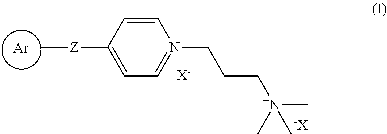
wherein
Ar comprises a triphenylamine group or a tetraphenylene group;
Z comprises a direct bond, an electron-rich π-conjugated unit, a benzothiadiazole, or a benzothiadiazole alkenyl group; and
X comprises a halogen or a derivative thereof.

2. The composition according to claim 1, wherein Ar comprises a triphenylamine group selected from a group consisting of:

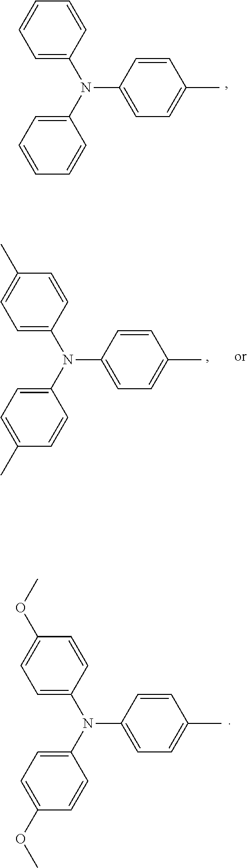 [SMILES: Cc1ccc(N(c2ccccc2)c2ccccc2)cc1 ; Cc1ccc(N(c2ccc(C)cc2)c2ccc(C)cc2)cc1 ; COc1ccc(N(c2ccc(C)cc2)c2ccc(OC)cc2)cc1]

3. The composition according to claim 1, wherein Ar comprises a tetraphenylene group selected from a group consisting of:

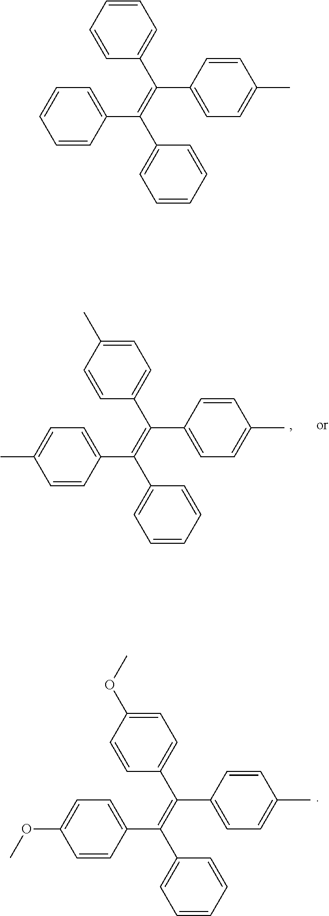

4. The composition according to claim 1, where Z comprises a benzothiadiazole vinyl group.

5. The composition according to claim 1, wherein X comprises bromine or a derivative thereof.

6. The composition according to claim 1, wherein the compound of Formula (I) is selected from a group consisting of:

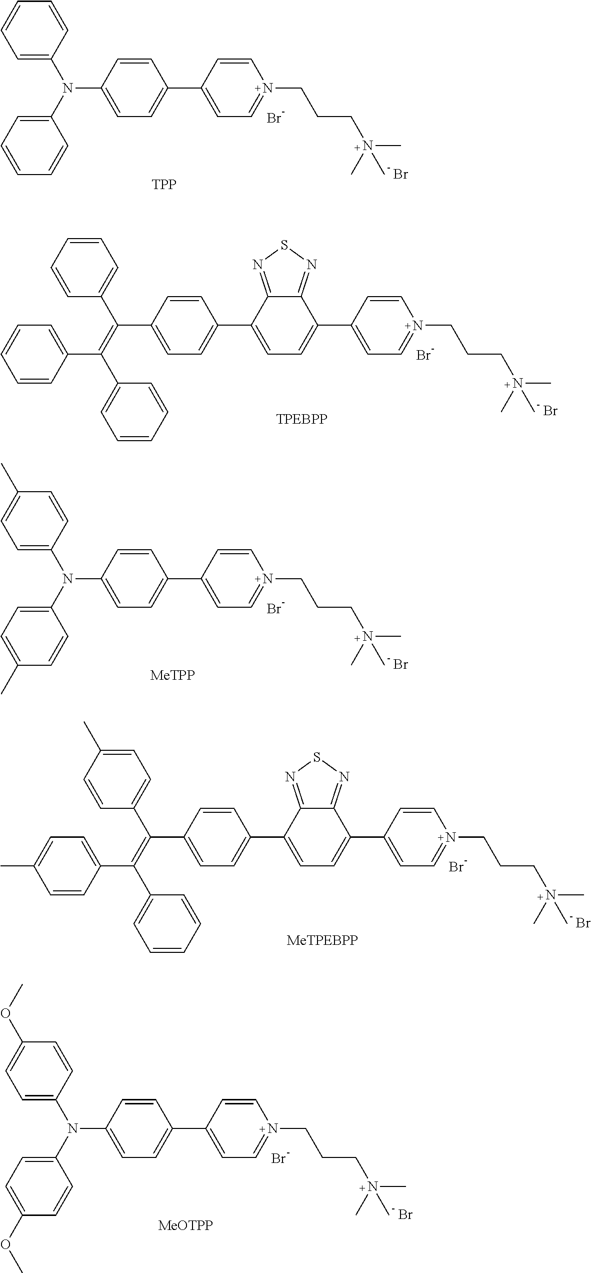

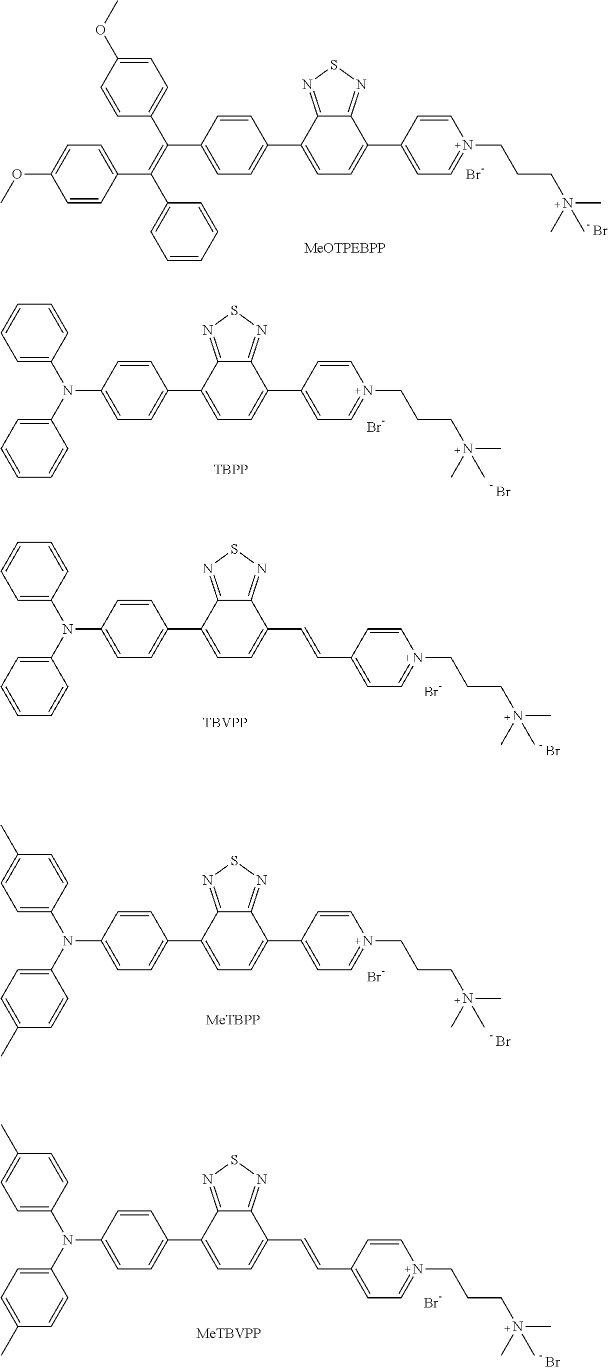

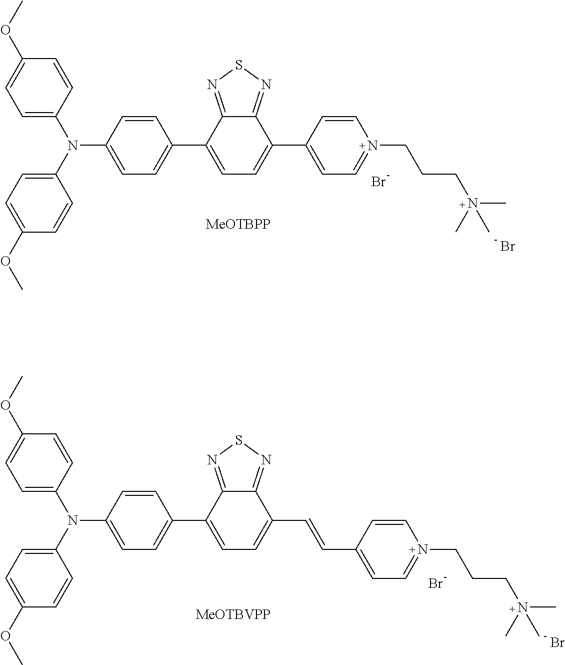

7. The composition according to claim 1, wherein the compound of Formula (I) is selected from a group consisting of:
4-(4-(diphenylamino)phenyl)-1-(3-(trimethylammonio)propyl)pyridin-1-ium bromide;
4-(4-(di-p-tolylamino)phenyl)-1-(3-(trimethylammonio)propyl)pyridin-1-ium bromide;
4-(4-(bis(4-methoxyphenyl)amino)phenyl)-1-(3-(trimethylammonio)propyl)pyridin-1-ium bromide;
1-(3-(trimethylammonio)propyl)-4-(7-(4-(1,2,2-triphenylvinyl)phenyl)benzo[c][1,2,5]thiadiazol-4-yl)pyridin-1-ium bromide;
(E)-4-(7-(4-(2-phenyl-1,2-di-p-tolylvinyl)phenyl)benzo[c][1,2,5]thiadiazol-4-yl)-1-(3-(trimethylammonio)propyl)pyridin-1-ium bromide;
(Z)-4-(7-(4-(1,2-bis(4-methoxyphenyl)-2-phenylvinyl)phenyl)benzo[c][1,2,5]thiadiazol-4-yl)-1-(3-(trimethylammonio)propyl)pyridin-1-ium bromide;
4-(7-(4-(diphenylamino)phenyl)benzo[c][1,2,5]thiadiazol-4-yl)-1-(3-(trimethylammonio)propyl)pyridin-1-ium bromide;
(E)-4-(2-(7-(4-(diphenylamino)phenyl)benzo[c][1,2,5]thiadiazol-4-yl)vinyl)-1-(3-(trimethylammonio)propyl)pyridin-1-ium bromide;
4-(7-(4-(di-p-tolylamino)phenyl)benzo[c][1,2,5]thiadiazol-4-yl)-1-(3-(trimethylammonio)propyl)pyridin-1-ium bromide;
(E)-4-(2-(7-(4-(di-p-tolylamino)phenyl)benzo[c][1,2,5]thiadiazol-4-yl)vinyl)-1-(3-(trimethylammonio)propyl)pyridin-1-ium bromide;
4-(7-(4-(bis(4-methoxyphenyl)amino)phenyl)benzo[c][1,2,5]thiadiazol-4-yl)-1-(3-(trimethylammonio)propyl)pyridin-1-ium bromide;
(E)-4-(2-(7-(4-(bis(4-methoxyphenyl)amino)phenyl)benzo[c][1,2,5]thiadiazol-4-yl)vinyl)-1-(3-(trimethylammonio)propyl)pyridin-1-ium bromide.

8. The composition according to claim 1, wherein the compound of Formula (I) is adapted to emit fluorescence.

9. The composition according to claim 8, wherein the compound of Formula (I) is adapted to exhibit aggregation-induced emission (AIE) of fluorescence.

10. The composition according to claim 6, wherein one or more of the compounds TPP, MeTPP, MeOTPP, TPEBPP, MeTPEBPP or MeOTPEBPP are adapted to emit fluorescence in a visible spectrum ranged from color green, yellow, orange to red; and wherein one or more of the compounds TBPP, TBVPP, MeTBPP, MeTBVPP, MeOTBPP or MeOTBVPP are adapted to emit fluorescence ranged from a visible color red to a near-infrared (NIR) region.

11. The composition according to claim 1, wherein the compound of Formula (I) is adapted to generate reactive oxygen species (ROS) under irradiation.

12. The composition according to claim 1, further comprises a therapeutic selected from a group consisting of anti-tumor therapeutic, cardiovascular therapeutic, respiratory therapeutic, digestive therapeutic, urinary infection therapeutic and oral infection therapeutic.

13. The composition according to claim 1, wherein the composition is for use in diagnosing and treating a bacterial infection in relation to one or more of cancer or cancer-related disease, cardiovascular disease, respiratory disease, digestive disease, urinary disease, and oral disease.

14. A method for diagnosing and treating a bacterial infection in a subject, comprising administering to the subject in need thereof an effective amount of the composition according to claim 1.

15. The method according to claim 14, further comprising one or more steps of:
binding the compound of Formula (I) with bacteria comprising planktonic bacteria and/or biofilm;
emitting fluorescence by the compound of Formula (I) at the bound bacteria; and/or
generating reactive oxygen species (ROS) under irradiation to thereby damage, inhibit growth of, and/or eliminate the bound bacteria.

16. The method according to claim 14, wherein the effective amount of the composition comprises around 1 μM to around 10 μM of the compound of Formula (I).

17. The method according to claim 15, further comprising administering to the subject in need thereof an effective amount of one or more therapeutics.

18. A combined diagnostic and therapeutic agent for treating a bacterial infection associated condition or disorder, comprising:
the composition according to claim 1, and one or more therapeutics.

19. The combined diagnostic and therapeutic agent according to claim 18, wherein the one or more therapeutics are selected from a group consisting of doxorubicin, aldesleukin, cisplatin, oxaliplatin, 5-fluorouracil, cytarabine, gemcitabine, and methotrexate.

20. The combined diagnostic and therapeutic agent according to claim 18, wherein the bacterial infection associated condition or disorder is selected from a group consisting of cancer or cancer-related disease, cardiovascular disease, respiratory disease, digestive disease, urinary disease and oral disease.
